(19) United States
(12) Patent Application Publication
(10) Pub. No.: US 2022/0298484 A1
(43) Pub. Date: Sep. 22, 2022

(54) ROLE OF CYR61 IN EXTRACELLULAR MATRIX FOR RETENTION OF MESENCHYMAL STEM CELL PROPERTIES

(71) Applicant: THE BOARD OF REGENTS OF THE UNIVERSITY OF TEXAS SYSTEM, Austin, TX (US)

(72) Inventors: Xiao-Dong CHEN, San Antonio, TX (US); MIlos MARINKOVIC, San Antonio, TX (US)

(73) Assignee: THE BOARD OF REGENTS OF THE UNIVERSITY OF TEXAS SYSTEM, Austin, TX (US)

(21) Appl. No.: 17/617,270

(22) PCT Filed: Jun. 4, 2020
(86) PCT No.: PCT/US20/36123

§ 371 Date: Dec. 7, 2021

Related U.S. Application Data
(60) Provisional application No. 62/858,767, filed on Jun. 7, 2019.

(51) Int. Cl. C12N 5/0775; A61K 35/28

(52) U.S. Cl. CPC C12N 5/0663; A61K 35/28; C12N 2533/90; C12N 2523/00; C12N 2500/32

(57) ABSTRACT

Disclosed are methods for restoring stem cell properties to stem cells in need thereof. Stem cells that have diminished or substantially no stem cell properties are cultivated on extracellular matrix that is produced by cells that are capable of producing CCN1/Cyr61 to produce a rescued stem cell culture. The rescued stem cell culture exhibits restored stem cell properties including responsiveness to differentiation inducers and/or adipogenesis inducers. The rescued stem culture can be used in autologous stem cell based therapies.

### CROSS REFERENCE TO RELATED APPLICATIONS

This Application claims the benefit of priority to U.S. Provisional Patent Application Ser. No. 62/858,767, filed Jun. 7, 2019, which is hereby incorporated by reference in its entirety.

### FIELD OF INVENTION

The present invention concerns a method for restoring stem cell properties. More specifically, the present invention concerns a method that uses an extracellular matrix comprising CCN1/Cyr61 to restore stem cell properties for mesenchymal stem cells.

### BACKGROUND OF THE INVENTION

Mesenchymal stem cells (MSCs), are multipotent stem cells that can differentiate into a number of types of cells including osteogenic (bone) cells, chondrogenic (cartilage) cells, myogenic (muscle) cells and adipogenic (fat) cells. Mesenchymal stem cells can be isolated from bone marrow, adipose tissue, umbilical cords, umbilical cord blood, placenta and amniotic fluid. Currently, MSCs are the subject of many research projects for treatment of numerous inflammatory and autoimmune conditions.

In vivo, MSCs mature, divide, and thrive within a microenvironment provided by the Extracellular Matrix (ECM). Replication of this niche in vitro results in higher quality MSCs. Researchers have discovered that MSCs cultured in vitro on ECM created by bone marrow stromal cells from an older donor have deficiency in self-renewal and differentiation, while cells cultured in vitro on ECM produced by stromal cells from a young donor have enhanced attachment, proliferation, and retention of stem cell properties.

For stem cell based therapies, autologous stem cell transplants are preferred over allogeneic (human leukocyte antigen (HLA) matched donor) transplants for several reasons, including reduced potential for immune reaction to transplanted materials (graft versus host disease), negating the need for anti-rejection therapies, and improvement of the overall success of the procedure. For instance, currently, allogeneic MSC culture systems, which use 3D scaffolds with added cytokines and nutrients to support the development of the MSCs, suffers a few drawbacks including variability of the scaffold due to the source of the MSCs and the preparation process, contamination from the MSC donors, variability in small concentration components that can significantly affect cell proliferation, cell differentiation, cell health, and contamination from xenographic components. However, many target conditions for stem cell therapies mainly affect older adults over younger adults and children. The decreased MSC quality/viability from older donors can make obtaining sufficient amount of healthy autologous stem cells challenging.

Overall, while methods for producing healthy stem cells and improving stem cell quality exist, the need for improvements in this field persists in light of at least the aforementioned drawbacks for the conventional methods.

### SUMMARY OF THE INVENTION

A solution to the above-mentioned problems associated with stem cell cultivation has been discovered. The solution resides in a method of restoring stem cell properties to stem cells in need thereof by cultivating the stem cells in extracellular matrix that is produced by cells capable of producing CCN1/Cyr61. This can be beneficial for at least restoring stem cell properties, including capability for differentiation and/or self-renewal, to stem cells in need thereof, e.g., stem cells from older donors. Thus, the discovered method is capable of improving the qualities of stem cells from older donors for stem cell therapies, and consequently producing autologous stem cell cultures from older donors whose original stem cells show diminished stem cell properties. Hence, the disclosed method may be able to facilitate autologous stem cell transplantation based therapies for older patients. Therefore, the methods of the present invention provide a technical solution to at least some of the problems associated with the conventional method for stem cell cultures and stem cell based therapies.

Some embodiments of the present invention are directed to a method of restoring stem cell properties to stem cells in need thereof. In some aspects, the method may comprise providing an extracellular matrix that is produced by cells capable of producing CCN1/Cyr61. The method may comprise cultivating the stem cells in need of restoring stem cell properties on the extracellular matrix under culture conditions sufficient to form a rescued stem cell culture. In some aspects, stem cells of the rescued stem cell culture exhibit restored stem cell properties.

Some embodiments of the present invention are directed to a method of restoring mesenchymal stem cell culture from stem cells in need of restoring stem cell properties. In some aspects, the method may comprise providing mesenchymal stem cells in need of restoring stem cell properties. The method may comprise producing extracellular matrix from bone marrow stromal cells that is capable of producing CCN1/Cyr61. The method can comprise cultivating the mesenchymal stem cells in need of restoring stem cell properties on the extracellular matrix under culture conditions sufficient to form a rescued mesenchymal stem cell culture. In some aspects, the mesenchymal stem cells of the rescued mesenchymal stem cell culture may exhibit restored stem cell properties including capability for differentiation, capability for self-renewal, viability, or combinations thereof.

Some embodiments of the present invention are directed to a method of treating an aged mesenchymal stem cells-related condition for an individual in need thereof. In some aspects, the method may comprise obtaining mesenchymal stem cells in need of restoring stem cell properties from the individual. The method may comprise producing extracellular matrix from bone marrow stromal cells that is capable of producing CCN1/Cyr61. The method may comprise cultivating the aged mesenchymal stem cells on the extracellular matrix under culture conditions sufficient to produce rescued mesenchymal stem cells with restored stem cell properties. The method may comprise administering the rescued mesenchymal stem cells to the individual at a dosage sufficient to alleviate the aged mesenchymal stem cell-related condition.

The terms “about” or “approximately” are defined as being close to as understood by one of ordinary skill in the art. In one non-limiting embodiment the terms are defined to be within 10%, preferably, within 5%, more preferably, within 1%, and most preferably, within 0.5%.

The terms “wt. %,” “vol. %,” or “mol. %” refers to a weight, volume, or molar percentage of a component, respectively, based on the total weight, the total volume, or the total moles of material that includes the component.

The term “substantially” and its variations are defined to include ranges within 10%, within 5%, within 1%, or within 0.5%.

The terms “inhibiting” or “reducing” or “preventing” or “avoiding” or any variation of these terms, when used in the claims and/or the specification, includes any measurable decrease or complete inhibition to achieve a desired result.

The term “effective,” as that term is used in the specification and/or claims, means adequate to accomplish a desired, expected, or intended result.

The use of the words “a” or “an” when used in conjunction with the term “comprising,” “including,” “containing,” or “having” in the claims or the specification may mean “one,” but it is also consistent with the meaning of “one or more,” “at least one,” and “one or more than one.”

The phrase “and/or” means and or or. To illustrate, A, B, and/or C includes: A alone, B alone, C alone, a combination of A and B, a combination of A and C, a combination of B and C, or a combination of A, B, and C. In other words, “and/or” operates as an inclusive or.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The process of the present invention can “comprise,” “consist essentially of,” or “consist of” particular ingredients, components, compositions, etc., disclosed throughout the specification. With respect to the phrase “consisting essentially of,” a basic and novel property of the compositions and methods of the present invention is the ability to restore stem cell properties.

Throughout this application, the MSCs and BM-MSCs include any progeny cells produced thereof. The term “progeny cell” is used to indicate a cell that is derived from another cell, such as a parent cell. The progeny cell may retain the same characteristics as the parent cell or may have different characteristics, such as a progeny cell that has differentiated.

Throughout this application, “decreased quantity and/or quality” of bone marrow-derived mesenchymal stem cells is used to indicate that the number of stem cells is decreased and/or stem cell function is diminished along one or more dimensions relative to those of a young, healthy subject population's. Non-limiting examples are shown herein of properties of stemness (i.e. SSEA-4, self-renewal, differentiation capacity) and properties of aging (senescence, reactive oxygen species, annexin-5). In a non-limiting example, aging can cause a decreased quantity and/or quality of bone marrow-derived mesenchymal stem cells.

Throughout this application, the term “aging” is used to indicated the sum of processes, by which stem cell populations decrease in quantity and/or quality.

Throughout this application, the term “young” refers to humans (male or female) age 25 years and under, and also refers to the cells obtained from them.

Throughout this application, the term “elderly”, “old”, or “older” refers to humans (male or female) age 65 years and older, and also refers to the cells obtained from them.

Throughout this application, the term “subject”, “patient”, or “donor” refers to a male or female human.

Other objects, features and advantages of the present invention will become apparent from the following figures, detailed description, and examples. It should be understood, however, that the figures, detailed description, and examples, while indicating specific embodiments of the invention, are given by way of illustration only and are not meant to be limiting. Additionally, it is contemplated that changes and modifications within the spirit and scope of the invention will become apparent to those skilled in the art from this detailed description. In further embodiments, features from specific embodiments may be combined with features from other embodiments. For example, features from one embodiment may be combined with features from any of the other embodiments. In further embodiments, additional features may be added to the specific embodiments described herein.

### DESCRIPTION OF THE DRAWINGS

For a more complete understanding, reference is now made to the following descriptions taken in conjunction with the accompanying drawings, in which:

FIGS. 1A to 1C show the results of effects of old- and young-ECMs on bone marrow mesenchymal stem cells (BM-MSCs) proliferation and response to BMP-2. FIG. 1A shows results for young BM-MSCs (P1) that were cultured for 7 days on tissue culture plastic (TCP) and young (Y)- or old (O)-ECM; proliferation was assessed by cell counts. *P<0.05 (n=3), vs. O-ECM or TCP. FIG. 1B shows results for young BM-MSCs that were cultured for 7 days and then treated with BMP-2 (60 ng/ml) in reduced serum media for 2 days. Runx2 expression was assayed by TaqMan PCR. *P<0.05 (n=3), vs. untreated (Unt.). FIG. 1C shows results for studies in young BM-MSCs cultivated on O-ECM that is made by cells from 3 different elderly donors. *P<0.05 (n=3), vs. untreated. The fold-change in Runx2 expression with BMP-2 treatment is shown in (FIG. 1B) and (FIG. 1C) and indicative of cell sensitivity;

FIGS. 2A to 2D show topographical and mechanical properties of young (Y) versus old (O)-ECM. FIG. 2A shows AFM images highlighting the topographic differences between Y-ECM vs. O-ECM. The scan area is 70×70 μm. FIG. 2B shows mean roughness maximal (Max.) height of Y- vs. O-ECMs. *β<0.05 (n=15), vs O-ECM. FIG. 2C shows maximal (Max.) height of Y- vs. O-ECMs. *β<0.05 (n=15), vs O-ECM. FIG. 2D shows mechanical properties of Y-, O- and AD-ECM (adipose tissue-derived extracellular matrix) were measured using small angle oscillatory shear (SAOS). *P<0.05 (n=4), vs. O- or AD-ECMs.

FIGS. 3A to 3C show results of concentrations of CCN1/Cyr61 in young (Y)- and old (O)-ECMs. FIG. 3A shows a Venn diagram for the differences/similarities in protein composition of Y-, O-, and AD-ECMs based on proteomic analyses. Each ECM sample consisted of a pooled lysate of matrices synthesized by cells from three individual donors; FIG. 3B shows CCN1/Cyr61 expression during ECM synthesis by old and young BM-derived stromal cells. FIG. 3C shows immunofluorescence confocal microscopy images for visualizing the presence of CCN1/Cyr61 protein in Y- and O-ECMs (25× mag). CCN1/Cyr61 staining was significantly brighter and more extensive in Y-ECM compared to O-ECM. (Mean gray value of 10 randomly selected areas: Y-ECM=137; O-ECM=81);

FIGS. 4A and 4B show results of Western blot analysis using cultures obtained from description of FIGS. 4A to 4C. FIG. 4A shows results when 80 μg protein/well was used for Western blot analysis with Cyr61: 42-53 kDa; FIG. 4B shows the results of quantification of band density.

FIGS. 5A and 5B show results of effects of CCN1/Cyr61 on MSC proliferation and responsiveness to BMP-2. FIG. 5A shows effect of exogenous CCN1/Cyr61 on the proliferation of MSCs cultured for 7 days on TCP versus young-ECM (Y-E). *P<0.05 (n=3), vs. treatment with 0 or 50 ng/m1 protein (50), or Y-E; FIG. 5B shows results for cells cultured for 7 days on TCP in the presence of varying doses of CCN1/Cyr61 or on Y-E alone and treated with BMP-2 for 2 days. Runx2 expression was determined by TaqMan PCR. *P<0.05 (n=3), vs. untreated (Unt.).

FIGS. 6A to 6C show results for knockdown and overexpression of CCN1/Cyr61 in young or old BM cells. FIG. 6A shows results of young cells (Y-C) with CCN1/Cyr61 expression silenced by siRNA treatment. At the same day in culture, CCN1/Cyr61 expression in siRNA-treated Y-C was lower than in old cells (O-C). Scrambled is a negative control for siRNA. *P or P<0.05 (n=3), vs day 8 or the other groups; FIG. 6B shows over-expression of CCN1/Cyr61 in Y-C and O-C with AdCCN1/Cyr61 treatment. Null is a negative control for the adenovirus. *P<0.05 (n=3), vs the other groups on day 9; P<0.05 (n=3), vs O-C/Ad at day 8 or 9; and *P<0.05 (n=3), vs Y-C at day 8 or 9; FIG. 6C shows Immunofluorescence confocal microscopy of cell-free young (Y-E) and old (O-E) ECMs at day 11, produced by Y-C or O-C with or without AdCyr61 or siRNA treatment. The right image for each group is a negative control (primary staining with non-specific isotype antibody).

FIG. 7 shows results of Western blot analysis of CCN1/Cyr61 protein in young- and old-ECMs made by young or old cells treated with siRNA or AdCCN1/Cyr61 (AdC61), respectively; and

FIGS. 8A to 8C shows results of responsivness of young MSCs to BMP-2 and RGZ with maintenance on young-ECM (Y-E), young-ECM depleted or enriched in CCN1/Cyr61 (6⁻Y-E or 6⁺Y-E, respectively) and old-ECM (O-E) or old-ECM with restored CCN1/Cyr61 (6⁺O-E). FIG. 8 shows Runx2 expression. *P<0.05 (n=3), vs untreated (Unt.); FIG. 8B shows BSP expression. *P<0.05 (n=3), vs untreated (Unt.); FIG. 8C shows PPARγ expression. *P<0.05 (n=3), vs untreated (Unt.). The fold-change with BMP-2 or RGZ over Unt controls represents the sensitivity of cell response.

### DETAILED DESCRIPTION OF THE INVENTION

Currently, stem cell transplants are often performed using allogeneic cells, as many conditions that can be treated by stem cell therapies preferentially affect older individuals, whose mesenchymal stem cells often show diminished stem cell properties. However, the allogeneic transplants of mesenchymal stem cells are prone to immune reaction, which can cause serious complications for the patients. Furthermore, to perform allogeneic stem cell transplant, anti-rejection therapies are needed, thereby increasing the costs and failure rate for stem cell based therapies. The present invention provides a solution to at least some of these problems. The solution is premised on a method of restoring stem cells to stem cells in need thereof by cultivating stem cells with diminished stem cell properties (e.g., stem cells from older donors/patients) on extracellular matrix produced by cells that are capable of producing CCN1/Cyr61. The resulting rescued stem cells, which are autologous to the stem cells in need of restoring stem cell properties, exhibit improved stem cell properties including capability of cell differentiation and/or self-renewal. Therefore, the rescued stem cells can be used in autologous stem cell transplants, leading to reduced risk for immune reaction, and negated need for anti-rejection therapies for stem cells based therapies. Furthermore, serial administration of the rescued autologous stem cells to a patient may gradually reverse the microenvironment of the stem cells in the patient being treated, thereby delaying the progression of aged stem cell-related diseases.

These and other non-limiting aspects of the present invention are discussed in further detail in the following sections.

### A. The Extracellular Matrix

Besides its obvious roles in determining the architecture and mechanical properties of tissues, the ECM greatly influences cell adhesion, migration, proliferation, differentiation, and survival. ECM modulates the bioactivities of growth factors and cytokines, such as transforming growth factor-β (TGF-β), tumor necrosis factor-α, and platelet-derived growth factor, by activating latent growth factors via proteolytic processing, by sequestering growth factors and hindering them from binding to their receptors, or by directly affecting receptor activity. Cells residing in the ECM not only receive ECM cues but also influence ECM signaling by secreting ECM components and by producing enzymes that cause proteolytic modification of proteins and growth factors in the ECM. The end result is a “give and take” relationship between cells and the ECM that defines cell behavior.

Regardless of tissue types, the ECM consists of collagen fibers, laminin polymers, cell adhesion proteins such as fibronectin, high molecular-weight proteoglycans, various growth factors that often exist in a latent or masked form, and members of the small leucine-rich proteoglycan (SLRP) family, mainly biglycan (bgn) and decorin (dcn) (Clark and Keating, 1995; Hocking et al., 1998; Lee et al., 1999). As might be expected from such a complex composition, the structure of the ECM in most tissues is not well understood. However, based on the studies of kidney basal lamina and ECM of skin, it is generally accepted that the ECM structure is dictated by the interaction of collagen fibers with each other and with laminin, as well as high-molecular-weight proteoglycans, resulting in the formation of an interlocking mesh-like structure (Pollard and Earnshaw, 2002). SLRPs such as bgn and dcn are also associated with collagen fibers and also with fibronectin and growth factors in the ECM. SLRPs appear to be important for collagen fibrillogenesis, as well as growth factor localization.

The loss of stemness during growth of MSCs using current culture methods reflects the production of more differentiated progeny with diminished self-renewal capacity, rather than the production of identical daughter stem cells. The term “stemness” refers to the stem cell properties including self-renewal (proliferation) and multipotentiality (capacity for the differentiation into multiple cell lineages). Involvement of the ECM in the regulation of mesenchymal colony forming units (MCFUs) is further supported by evidence that deletion of the ECM components biglycan and decorin has a deleterious effect on responsiveness of marrow derived osteoblast progenitors to BMPs and TGF-β (Di Gregorio et al., 2001; Chen et al., 2004). At this stage, it is unknown how the ECM regulates the behavior of MCFUs. Earlier work has shown that the ECM modulates the activity of growth factors by controlling proteolytic activation of latent factors, as occurs in the case of TGF-β (Dallas et al. 2002). The ECM also interacts with cell surface receptors to prevent binding of the cognate ligand, as occurs in the case of the epidermal growth factor (EGF) receptor (Santra et al., 2002), and sequesters factors such as platelet-derived growth factor (PDGF) and BMPs (Suzawa et al., 1999; Nili et al., 2003). The ECM may also bind growth-promoting factors from the serum for optimal presentation to MSCs. Finally, the ECM may enhance the function of putative accessory cells that support MCFU replication.

### B. Method Of Restoring Stem Cell Properties

Autologous stem cells are preferential for stem cell based treatments over allogeneic (HLA matched donor) stem cells. However, the stem cells from the individual in need of these stem cell based treatments often exhibit reduced or diminished stem cells properties, resulting in challenges to obtain fully healthy autologous stem cells. The method disclosed herein is capable of facilitating production of autologous stem cell cultures using stem cells having reduced or diminished stem cell properties.

Embodiments of the invention include a method for restoring stem cell properties to stem cells in need thereof. In some aspects, the stem cells in need of restoring stem cell properties can include stem cells with decreased quality of stem cells. In some instances, the stem cells in need of restoring stem cell properties can include aged stem cells that have reduced or substantially no stem cell properties compared to young stem cells. In some aspects, the stem cells in need of restoring stem cell properties may include stem cells from an individual over an age of 55 years. In some aspects, the stem cells in need of restoring stem cell properties may include stem cells from an individual at an age of 55 to over 96 years old. Non-limiting examples of the stem cells include mesenchymal stem cells derived from various tissues including bone marrow, adipose tissue, cartilage tissue, or any combination thereof. In some aspects, the mesenchymal stem cells may include bone marrow mesenchymal stem cells, adipose mesenchymal stem cells, umbilical cord mesenchymal stem cells, or any combinations thereof.

In some aspects, the properties of stem cells may include capability for differentiation, capability for self-renewal, viability or any combination thereof. In some instances, the stem cells in need of restoring stem cell properties may have substantially no response to an differentiation inducer. In some instances, the differentiation inducer may include an osteoblastogenesis inducer, an adipogenesis inducer, a chondroblastogenesis inducer, a musclegenesis inducer, or any combination thereof. Non-limiting examples of the osteoblastogenesis inducer may include BMP-2. In some instances, the stem cells in need of restoring stem cell properties may have substantially no response to an adipogenesis inducer. Non-limiting examples of adipogenesis inducer may include rosiglitazone

In some instances, the response of stem cells in need of restoring stem cell properties to an osteoblastogenesis inducer (e.g., BMP-2) can be determined by treating the stem cells with 10 to 100 ng/ml of the osteoblastogenesis inducer for 1 to 7 days and testing Runx2 expression levels after the treatment of the osteoblastogenesis. In some aspects, there is substantially no increase in Runx2 expression level of the osteoblastogenesis inducer treated stem cells in need of restoring stem cell properties, as compared to Runx2 expression level for stem cells that are from the same donor but not treated with osteoblastogenesis inducer, thereby indicating substantially no response to the osteoblastogenesis inducer.

In some instances, the response of stem cells in need of restoring stem cell properties to an adipogenesis inducer (e.g., rosiglitazone) can be determined by treating the stem cells with about 1.9 μg/ml of the adipogenesis inducer for about 2 days and testing peroxisome proliferator-activated receptor gamma (PPARγ) expression levels after the treatment of adipogenesis. In some aspects, there is substantially no increase in PPARγ expression level of the adipogenesis inducer treated stem cells in need of restoring stem cell properties, as compared to PPARγ expression level for stem cells that are from the same donor but not treated with the adipogenesis inducer, thereby indicating substantially no response to the adipogenesis inducer.

In embodiments of the invention, the method may include providing stem cells in need of restoring stem cell properties. In some aspects, the stem cells in need of restoring stem cell properties produce extracellular matrix that is deficient in CCN1/Cyr61 protein. In some instances, the stem cells in need of restoring properties is obtained from bone marrow of an individual that is more than 65 years old.

In embodiments of the invention, the method may include providing an extracellular matrix that is produced by cells capable of producing CCN1/Cyr61. In some aspects, the cells capable of producing CCN1/Cyr 61 may include marrow stromal cells, skin cells, muscle cells, or any combination thereof. In some instances, the cells capable of producing CCN1/Cyr61 include cells that are from an individual no more than 65 years old. In some instances, the cells capable of producing CCN1/Cyr61 include cells that contain recombinant DNA fragments for CCN1/Cyr61 expression. In some aspects, the extracellular matrix comprises CCN1/Cyr61 and other extracellular matrix proteins including fibronectin, collagen, RGD-CAP/Betaig-h3, EMILIN-1, periostin, biglycan, thrombospondin-1, tenascin, heparin sulfate, proteoglycan, fibulin-1, galectin-1, decorin, lumican, fibulin-2, ostenectin, fibrillin-2, or any combinations thereof.

In some aspects, the extracellular matrix can be produced by cultivating the cells capable of producing CCN1/Cyr61 in a culture media for 5 to 7 days to form a cell culture mixture containing the extracellular matrix. In some aspects, the produced extracellular matrix can be harvested from cell culture mixture by decellularization using 0.5% Triton X-100 containing 20 mM NH₄OH in PBS. In some aspects, presence of CCN1/Cyr61 in the produced extracellular matrix may be confirmed by reverse transcription polymerase chain reaction (RT-PCR) at RNA levels, and/or immunofluorescence staining and Western Blot analysis at protein levels.

In some aspects, CCN1/Cyr61 may be a cysteine-rich protein coded by a serum-inducible immediate-early gene. In some aspects, gene structure for coding CC1/Cyr61 contains 5 exons and 4 introns with each exon coding for a modular domain with sequence homology to insulin-like growth factor binding proteins (IGFBP), the von Willebrand factor C (VWC) domain, thrombospondin type 1 (TSP-1) domain, and a carboxyl-terminal domain that contains a cysteine knot motif. In some aspects, CC1/Cyr61 may have cell type specific and context sensitive effects.

In embodiments of the invention, the method may include cultivating the stem cells in need of restoring stem cell properties on the extracellular matrix under culture conditions sufficient to multiply the stem cells and form a rescued stem cell culture that exhibits restored stem cell properties. In some aspects, the culture conditions may include a culture temperature of about 37° C. In some aspects, the culture conditions may include an ambient atmospheric carbon dioxide concentration of about 5 vol. %. In some aspects, the culture conditions may include αMEM (minimum essential medium) supplemented with glutamine and 15% fetal bovine serum. In some aspects, the culture conditions may include cultivating the stem cells on tissue culture plastic (TCP).

In some aspects, the rescued stem cell culture having restored stem cell properties (e.g., improved stem cell quality) is from the same donor whose stem cells (before the rescuing) are in need of restoring stem cell properties. Therefore, in some aspects, the rescued stem cell culture may be suitable for autologous cell based-therapies. In some instances, the autologous stem cell based therapies may be adapted to treat conditions including osteoarthritis, general injury, graft versus host disease, lupus, multiple sclerosis, rheumatoid arthritis, Type I diabetes, or any combinations thereof.

In embodiments of the invention, the rescued stem cell may be used in a method of treating an aged mesenchymal stem cell-related condition for an individual in need thereof. The method may include administering the rescued stem cell culture, including rescued mesenchymal stem cells, to the individual at a dosage sufficient to alleviate the aged mesenchymal stem cell related condition. In some aspects, the mesenchymal stem cells from the individual in need of the treatment of an aged mesenchymal stem cell-related condition may not be able to produce sufficient CNN1/Cyr61 in the extracellular matrix to restore stem cell properties to autologous mesenchymal stem cells. The administering of the rescued stem cell culture may be adapted to reverse the microenvironment of mesenchymal stem cells of the individual, delay progression of aging-related diseases for the individual, delay aging process of the individual, or any combination thereof. In some instances, the aging-related disease may include cardiovascular disease, cerebrovascular disease, high blood pressure, cancer, type 2 diabetes, Parkinson's disease, Alzheimer's disease, chronic obstructive pulmonary disease, osteoarthritis, osteoporosis, age-related macular degeneration, hearing loss, or any combination thereof.

In embodiments of the invention, the stem cells in need of restoring stem cell properties and the cells capable of producing CCN1/Cry6 may be from any mammal. The rescued stem cell culture may be used for autologous stem cell based therapy for the mammal. In some instances, the mammal may include a mouse, the stem cell in need of restoring stem cell properties may include may be mesenchymal stem cells from a mouse older than 18 months. The stem cell capable of producing CCN1/Cry6 may be bone marrow stromal cells from a mouse younger than 3 months.

As part of the disclosure of the present invention, specific examples are included below. The examples are for illustrative purposes only and are not intended to limit the invention. Those of ordinary skill in the art will readily recognize parameters that can be changed or modified to yield essentially the same results.

### EXAMPLE 1

### Evaluation of Effects of Old-ECM on Bone Marrow MSCs

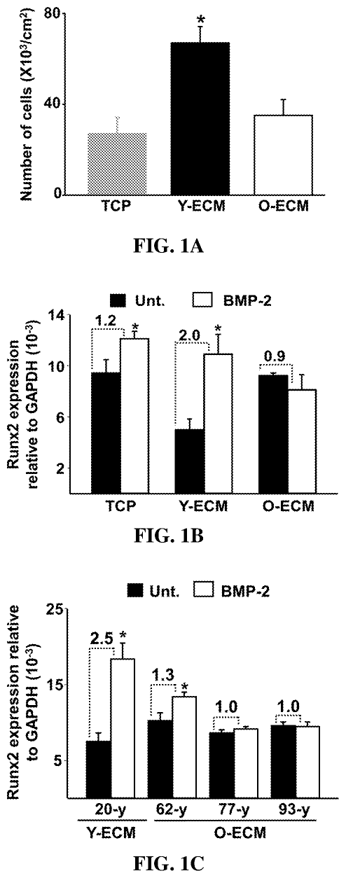

After 7 days in culture, proliferation of young BM-MSCs (from individual(s) between 18 to 23 years old) maintained on old-ECM (produced by bone marrow stromal cells from 72 years old individual) and TCP was 45-50% less than young BM-MSCs maintained on young-ECM (FIG. IA). Since response of MSCs to growth factors is largely influenced by the surrounding microenvironment, BMP-2 responsiveness experiments were performed in parallel with the proliferation studies. Young BM-MSCs, maintained for 7 days on old-ECM, did not respond to BMP-2 (60 ng/ml) treatment with increased Runx2 expression (a transcription factor for osteoblastic differentiation) (FIG. 1B), while MSCs maintained on young-ECM and TCP displayed increased Runx2 expression, 2- and 1.2-fold respectively.

To determine the reproducibility of these observations, the assays were prepared using old-ECM (produced by bone marrow stromal cells from randomly selected elderly donors of varying ages (62, 77, & 93 years old) (FIG. 1C). The results suggest that there is an age-dependent loss of BMP-2 responsiveness indicating the loss of key effective ECM components during aging.

### EXAMPLE 2

### Evaluation of Topographical and Mechanical Properties of Young and Old ECMs

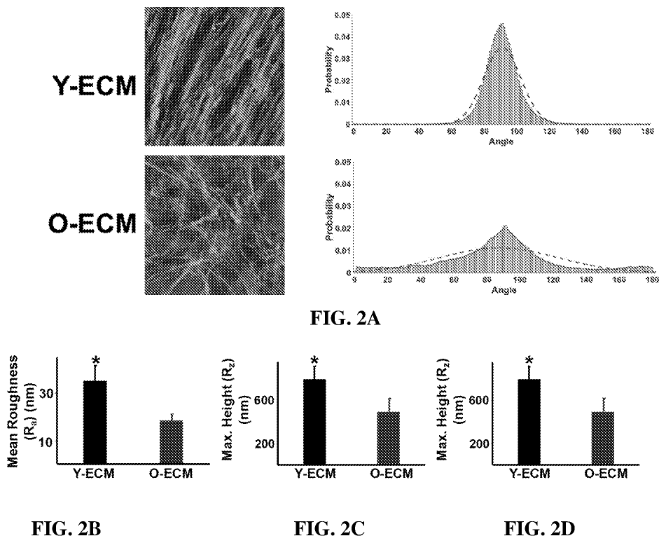

In addition to the functional differences described above, young- and old-ECMs have shown differences in both topographical and mechanical properties. These differences are known to influence MSC attachment, shape, motility, and differentiation. By atomic force microscopy (AFM), young-ECM was found to contain densely-organized and highly-oriented fibers (σ=11.3), while old-ECM was less densely-organized and had a broader range of orientations (σ=35.6) (FIG. 2A). Measurements of fiber orientation were performed on AFM images of 15 randomly-selected areas of young- and old-ECMs (70 μm×70 m). The data were fit to a normal distribution, with 90° corresponding to the mode of the observed orientations (FIG. 2A, right). Young-ECM had a mean Ra of ˜30 nm and Rz of ˜600 nm, which were higher than observed with old-ECM (FIGS. 2B and 2C). ECM mechanical characterization was performed using small angle oscillatory shear (SAOS) rheology. Young- and old-ECMs were significantly different in stiffness (FIG. 2D). Since adipose-derived (AD)-ECM (made by adipose-derived stromal cells from 20-year old donors) has architecture similar to that of old-ECM, its mechanical properties were measured as well. The differences between young- and old-ECMs may play a role in MSC differentiation.

### EXAMPLE 3

### Protein Composition Comparison between Young and Old ECMs

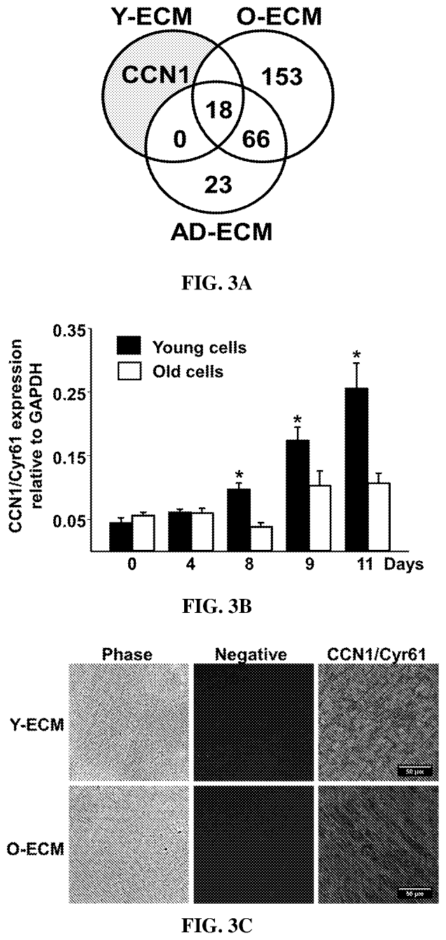

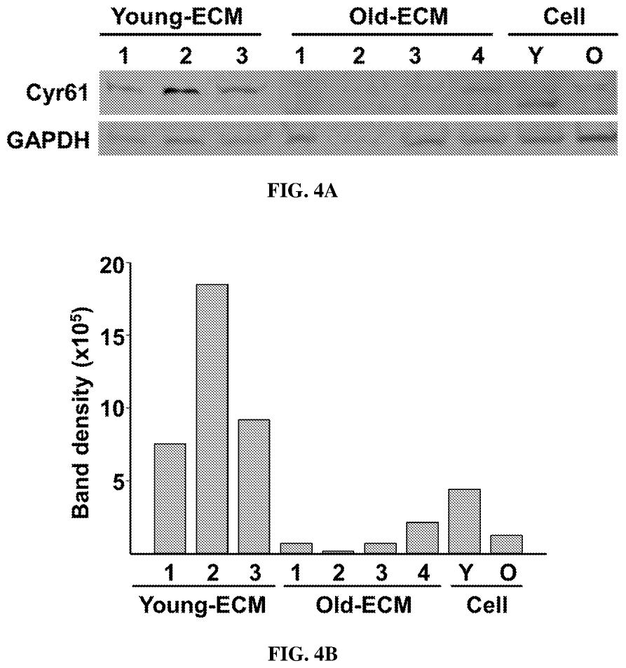

To further understand the molecular mechanisms responsible for the different properties of young-ECM versus old-ECM, the protein compositions of the two types ECMs were analyzed and compared using mass spectrometry (MS/MS) (FIG . 3A). For these analyses, AD-ECM was also included to guide the differentiation of BM-MSCs to adipogenesis and further help identify the unique composition of the two BM-ECMs. The results show that the protein composition of old-ECM was relatively similar to AD-ECM and contained 66 unique proteins not found in young-ECM. In contrast, there were no proteins only shared between young- and AD-ECMs and only 18 proteins in common among young-, old-, and AD-ECMs. These 18 shared proteins displayed statistically significant differences (i.e. either higher or lower) among these ECMs. Importantly, CCN1/Cyr61 was the only protein in young-ECM but not found in old- or AD-ECM. To confirm this finding, CCN1/Cyr61 mRNA expression during ECM synthesis was measured and the results show that CCN1/Cyr61 expression by young stromal cells was significantly higher on days 8, 9, and 11 compared to old stromal cells (FIG. 3B). This experiment was repeated three times using cells from three different donors. These results were validated with immunofluorescence confocal microscopy and Western blot analysis. (FIGS. 3C, 4A and 4B).

Western blot analysis was performed on decellularized Y- and O-matrices made by cells randomly selected from 3 young (20 to 23 years old)- and 4 elderly-donors (93-, 77-, 72-, and 62-year old) to demonstrate the reproducibility of the differences in CCN1/Cyr61 protein deposition in young- and old-ECM (FIGS. 4A and 4B). Only old-ECM made by cells from a 62-year old donor showed a slight band (much less than those of young-ECM), which may explain why it retained the ability to support BMP-2 responsiveness when young-MSCs were maintained on this old-ECM (FIG. 1C). To confirm the origin of the CCN1/Cyr61 band on the blots, the protein in cell lysates from both young (Y) and old (O) donors were measured and the results show that they contained much less CCN1/Cyr61 than young-ECM. However, cell lysates from both young and old cells contained more GAPDH than the ECM. Some of the GAPDH found in the ECMs was likely due to cellular contamination during processing.

### EXAMPLE 4

### Effects of Exogenous CCN1/Cyr61 on MSCs

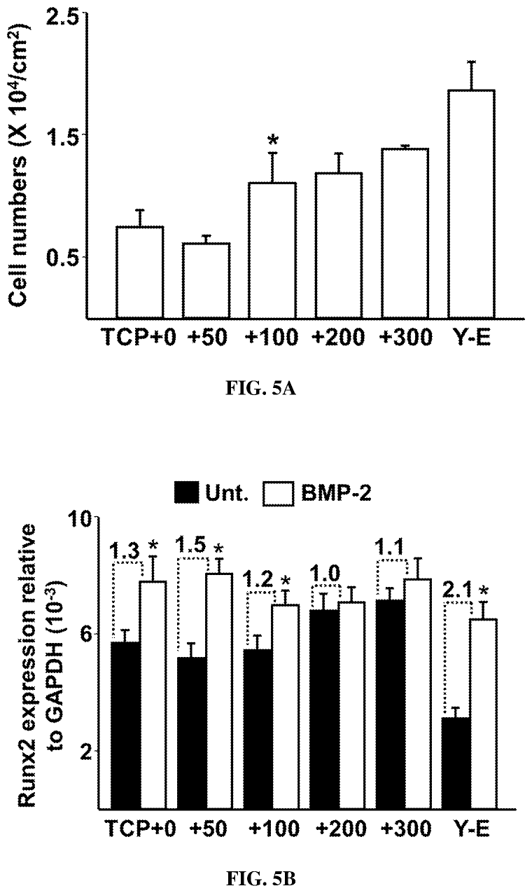

To test whether exogenous CCN1/Cyr61 influences MSC behavior, young BM-MSCs (Passage 1) were cultured for 7 days on TCP and then treated with varying doses (50-300 ng/ml) of recombinant human CCN1/Cyr61 (FIG. 5A). Cells treated with 100 ng/m1 of the protein grew significantly faster than untreated cells or those treated with 50 ng/ml; however, at doses >100 ng/m1 no further stimulation of cell growth was observed. In all cases, proliferation was less than that found on young-ECM alone. Compared to previous reports using mouse cell lines (e.g. stem cells [C3H10T1/2 and C2Cl2] or osteoprogenitor cells [MC-3T3-E1]), the effect of CCN1/Cyr61 on the proliferation of human primary BM-MSCs was less pronounced, suggesting that human primary cells may not be particularly sensitive to the exogenous protein. In parallel experiments, MSCs were treated with BMP-2 (60 ng/ml) after 7 days in culture on TCP (FIG. 5B) and the results show exogenous CCN1/Cyr61 did not have additional effect of CCN1/Cyr61 on responsiveness to BMP-2. In contrast, young MSCs maintained on young-ECM alone exhibited the highest sensitivity to BMP-2 treatment of all culture conditions tested.

The baseline of Runx2 expression appeared to increase with increasing doses of CCN1/Cyr61 (FIG. 5B), suggesting that higher doses might induce osteoblast differentiation. Based on these results, the concentration of 100 ng/ml for CCN1/Cyr61 was selected as the appropriate dose for further experiments.

### EXAMPLE 5

### Evaluation on Feasibility of Knockdown and Over-expression of CCN1/Cyr61

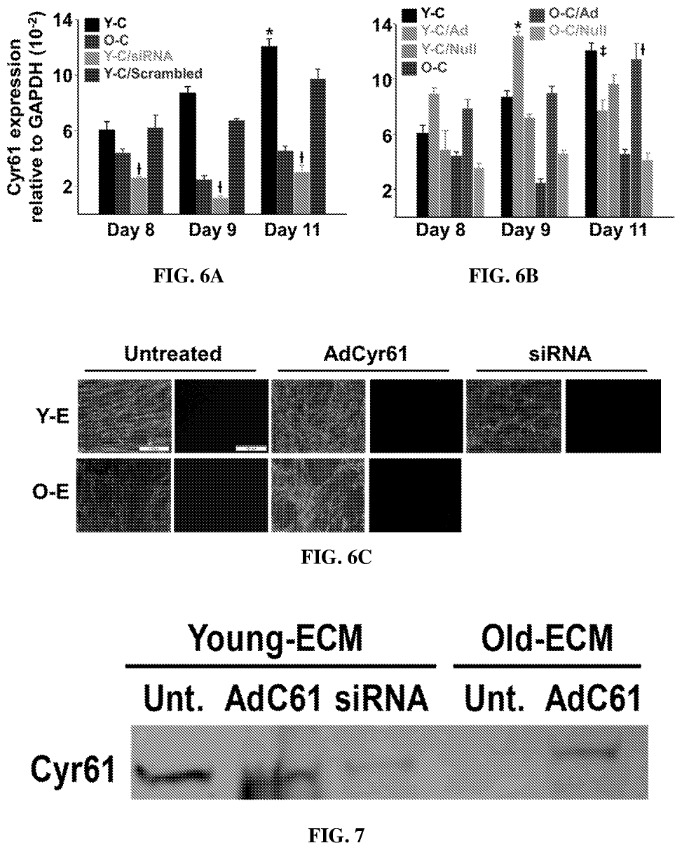

Young-BM stromal cells and old-BM stromal cells (Passage 1) were cultured for 5 days (˜70% confluence) and then treated with siRNA for 48 hours or adenovirus (AdCCN1/Cyr61) for 72 hours. On day 8, media containing 50 μM ascorbic acid were added and the cultures continued through day 11. CCN1/Cyr61 mRNA expression was measured using TaqMan PCR on day 8(before addition of ascorbate) and on day 9 and 11 (FIGS. 6A-6C). Treatment of young cells with siRNA successfully knocked down CCN1/Cyr61 expression (with or without ascorbate addition) as compared to untreated young (positive) or old (negative) cells (FIG. 6A). Furthermore, treatment with AdCCN1/Cyr61 promoted over-expression of the protein in young cells and restoration of expression in old cells (FIG. 6B).

Young cells infected with AdCCN1/Cyr61 displayed increased sensitivity to ascorbic acid stimulation, showing a peak of CCN1/Cyr61 expression on day 9 (FIG. 6B). The PCR results were confirmed by measuring CCN1/Cyr61 protein in the ECMs using immunofluorescence confocal microscopy (FIG. 6C) and Western blot analysis (FIG. 7) of day 11 cultures.

### EXAMPLE 6

### Evaluation of Effect of CCN1/Cyr61 on Responsiveness of MSCs to BMP-2 of MSCs

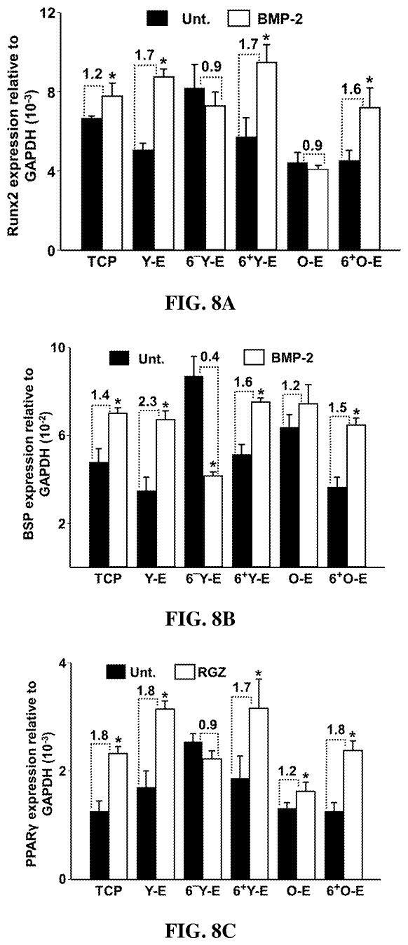

As described above (FIGS. 6A-6C), 1) CCN1/Cyr61-deficient young ECM (6-Y-E) by treating young cells with siRNA, 2) CCN1/Cyr61-overexpressed young-ECM (6+Y-E) and old-ECM (6+O-E) by infecting young and old cells with AdCCN1/Cyr61 were prepared, during ECM production. Young MSCs (Passage 1) were maintained for 7 days on TCP, Y-ECM (Y-E), 6-Y-E, 6+Y-E, O-ECM (O-E), or 6+O-E and then treated with BMP-2 (60 ng/ml) or RGZ (1 mg/ml) for 48 hrs in low-serum media. BMP-2 responsiveness was assayed by measuring Runx2 and bone sialoprotein protein (BSP, osteoblast marker) expression (FIGS. 8A and 8B), while RGZ (an inducer of adipogenesis) responsiveness was assayed by measuring PPARγ (transcription factor for adipogenesis) expression (FIG. 8C).

The results show that young-MSCs maintained on 6-Y-E lost responsiveness to both BMP-2 and RGZ based on smaller fold-changes in the expression of Runx2, BSP, and PPARγ than cells maintained on native Y-E. Moreover, the ability of O-E to retain MSC sensitivity to BMP-2 and RGZ was significantly rescued by incorporation of CCN1/Cyr61 into the matrix (i.e. 6+O-E). Further, it was unexpected that BSP expression would be higher in untreated young-MSCs, maintained on 6-Y-E, than with BMP-2 treatment, suggesting that the loss of CCN1/Cyr61 from the matrix resulted in an inability to retain MSC properties. The experiments were repeated four times, with cells from four different donors, and the same results were shown each time (data not shown). Overall, these results clearly suggest that CCN1/Cyr61 in the ECM plays a critical role in the retention of MSC properties and differentiation capacity.

Although embodiments of the present application and their advantages have been described in detail, it should be understood that various changes, substitutions and alterations can be made herein without departing from the spirit and scope of the embodiments as defined by the appended claims. Moreover, the scope of the present application is not intended to be limited to the particular embodiments of the process, treatment, machine, manufacture, composition of matter, means, methods, and/or steps described in the specification. As one of ordinary skill in the art will readily appreciate from the above disclosure, processes, machines, manufacture, compositions of matter, means, methods, or steps, presently existing or later to be developed that perform substantially the same function or achieve substantially the same result as the corresponding embodiments described herein may be utilized. Accordingly, the appended claims are intended to include within their scope such processes, machines, manufacture, compositions of matter, means, methods, or steps.

1. A method of restoring stem cell properties to stem cells in need thereof, the method comprising:
providing an extracellular matrix that is produced by cells capable of producing CCN1/Cyr61; and
cultivating the stem cells in need of restoring stem cell properties on the extracellular matrix under culture conditions sufficient to multiply the stem cells and form a rescued stem cell culture;
wherein the stem cells of the rescued stem cell culture exhibit restored stem cell properties.

2. The method of claim 1, wherein the stem cells include mesenchymal stem cells.

3. The method of claim 1, wherein the cells capable of producing CCN1/Cyr61 include bone marrow stromal cells, adipose-derived cells, muscle cells, or combinations thereof.

4. The method of claim 3, wherein the cells capable of producing CCN1/Cyr61 include cells that are from an individual no more than 65 year old.

5. The method of claim 3, wherein the cells capable of producing CCN1/Cyr61 include cells containing recombinant DNA fragments for CCN1/Cyr61 expression.

6. The method of claim 1, wherein the extracellular matrix comprises CCN1/Cyr61, fibronectin, collagen, periostin, biglycan, thrombospondin-1, or any combinations thereof.

7. The method of claim 1, wherein the culture conditions include a temperature in a range of about 37° C.

8. The method of claim 1, wherein the culture conditions include an ambient atmosphere having a carbon dioxide concentration of about 5 vol. %.

9. The method of claim 1, where in the culture conditions include cultivating on a tissue culture plastic.

10. The method of claim 1, wherein the culture conditions include aMEM (minimum essential medium) supplemented with glutamine and 15% fetal bovine serum.

11. The method of claim 1, wherein the properties of stem cells include capability for differentiation, capability for self-renewal, viability, or any combination thereof.

12. The method of claim 1, wherein the stem cells in need of restoring stem cell properties have substantially no response to an osteoblastogenesis inducer.

13. The method of claim 12, wherein the osteoblastogensis inducer includes BMP-2.

14. The method of claim 1, wherein the stem cells in need of restoring stem cell properties have substantially no response to an adipogensis inducer.

15. The method of claim 14, wherein the adipogensis inducer includes rosiglitazone.

16. The method of claim 1, wherein the rescued cell culture is suitable for autologous cell-based therapies.

17. A method of restoring stem cell properties to mesenchymal stem cells in need thereof, the method comprising:
providing mesenchymal stem cells in need of restoring stem cell properties;
providing extracellular matrix from bone marrow stromal cells that is capable of producing CCN1/Cyr61; and
cultivating the mesenchymal stem cells in need of restoring stem cell properties on the extracellular matrix under culture conditions sufficient to produce a rescued mesenchymal stem cell culture;
wherein the mesenchymal stem cells of the rescued mesenchymal stem cell culture exhibit restored stem cell properties including capability for differentiation, capability for self-renewal, viability, or any combination thereof.

18. A method of treating an aged mesenchymal stem cell-related condition for an individual in need thereof, the method comprising:
obtaining rescured mesenchymal stem cells with restored stem cell properties that have been cultivated on an extracellular matrix that is capable of producing CCN1/Cyr61; and
administering the rescued mesenchymal stem cells to the individual at a dosage sufficient to alleviate the aged mesenchymal stem cell-related condition.

19. The method of claim 18, wherein the aged mesenchymal stem cell related condition includes cardiovascular disease cerebrovascular disease, high blood pressure, cancer, type 2 diabetes, Parkinson's disease, Alzheimer's disease, chronic obstructive pulmonary disease, osteoarthritis, osteoporosis, age-related macular degeneration, hearing loss, or any combination thereof.
